(19) United States
(12) Patent Application Publication
(10) Pub. No.: US 2026/0034347 A1
(43) Pub. Date: Feb. 5, 2026

(54) Sensory Control System

(71) Applicant: Steve O'Neal, Oak Creek, CO (US)

(72) Inventor: Steve O'Neal, Oak Creek, CO (US)

(21) Appl. No.: 19/351,306

(22) Filed: Oct. 7, 2025

Related U.S. Application Data
(63) Continuation-in-part of application No. 18/766,408, filed on Jul. 8, 2024, now abandoned.
(60) Provisional application No. 63/525,940, filed on Jul. 10, 2023.

(51) Int. Cl. A61N 1/04; A61N 1/36

(52) U.S. Cl. CPC A61N 1/0456; A61N 1/0484; A61N 1/36034

(57) ABSTRACT

A sensory control system that encompasses an outer surface of a body part for applying low level electrical impulses to the outer surface to alter a physical sensation of the body part. The system including a garment constructed of a flexible material with an electrode disposed between the garment and the outer surface, wherein operationally the electrode comes into contact with an outer surface of a particular part of a human body of a user. Also included in the system is control circuitry that is in electrical communication with the electrode, wherein the control circuitry is operative to generate the electrical impulses with particular frequency, phase, pulse versus time amounts, voltage, and ampere levels at user safe low electrical power levels that are less than one-hundred milli-amps and no more than sixty volts.

### CROSS-REFERENCE TO RELATED APPLICATIONS

This application is a continuation in part of U.S. patent application Ser. No. 18/766,408 filed on Jul. 8, 2024, by Steve O'Neal of Oak Creek, Colorado, U.S., that claims the benefit of U.S. provisional patent application Ser. No. 63/525,940 filed on Jul. 10, 2023, by Steve O'Neal of Oak Creek, Colorado, U.S.

### BACKGROUND OF INVENTION

The present invention relates generally to a system for sensory control using electrical signals. More specifically, the present invention relates to the field of various devices using Transcutaneous Electrical Nerve Stimulation (TENS) having the advantages of being non-invasive and portable, TENS is a low voltage electric current that is in contact with the user's skin usually through an adhesive patch electrode with low electrical power levels (typically using smaller batteries) that can be varied in frequency from 1 to 120 hertz, wherein the TENS is used to help block nerve cell transmission of pain sensation or raising the level of pain blocking endorphins, being in either case to reduce the sensation of pain. Users report lasting effect of TENS can be zero, i.e. with the electrical current being removed the pain returns and users also report up to a twenty-four hour period of sensed pain reduction from TENS after removal.

The main reported risk of TENS is from skin irritation from the adhesive patch electrode, other risks that are associated with anybody can have issues with electrical impulses such as pacemakers, pregnancy, cancer, epilepsy, blood clots (as blood circulation is increased by TENS), infected areas, near eyes or mouth, near genitals, or low sensation areas. Skin contact can also be with acupuncture needles. Unique characteristics of a TENS system will be in novel skin contact structures (various electrode types) along with various body-hugging structures (garments) to hold the TENS system electrodes in the desired position on or adjacent to the body.

In looking at the prior art in the TENS field, starting with U.S. Pat. No. 8,421,448 to Tran, that discloses a glove with finger sensors for gesture commands, thus not being TENS, but showing patent teaching on glove fingertip embedded electrodes.

Next, in United States Patent Application Publication Number US2011/0007035 to Shai, discloses small elastomeric wearables that contain various sensors and further in United States Patent Application Publication Number US2015/0140934 to Abdurrahman, discloses wrist electrical wearable that wirelessly control through motion signals.

Continuing in U.S. Pat. No. 11,247,053 to Rajguru, discloses a TENS device for pain mitigation for foot, knee, ankle, and various other parts of the body, the novelty includes a stimulator electrode with signal control effectuated through sensory feedback, wherein the novelty is with the signal control and feedback system.

In addition, in U.S. Pat. No. 11,235,142 to Gozani, discloses a TENS smart electrode in the form of a wrap strap for a body wearable with novelty in the electrode having a controller that senses proper skin contact, a timer/intensity curve of the signal, and an electrode replacement notice.

Further, in U.S. Pat. No. 11,213,679 to Chen, discloses a portable TENS device that has Bluetooth to a smartphone app for control of battery state, signal level, signal strength limits, plus smartphone generated audio signals.

Moving onward, in U.S. Pat. No. 11,997,998 to Simon, discloses a headache pain electrical impulse treatment system via an electrode to the neck of a user that wirelessly receives a pain treatment regimen from a mobile device.

Next, U.S. Pat. No. 10,898,718 to Srivastava, discloses a sensor based pain management system having closed loop control for pain therapy, using two sensors and two circuits with a controller, this is to activate pain therapy based upon another user movement or event.

Further, in U.S. Pat. No. 10,661,072 to Kern, discloses a TENS attachment with a handheld pistol grip attachment electrode that converts mechanical motion to an electrical current to deliver the TENS therapy, also in U.S. Pat. No. 9,675,801 to Kong, discloses a TENS system with an electrode that is time measured to minimize skin irritation.

Continuing, in U.S. Pat. No. 8,868,216 to Dunagan, discloses a TENS system with an electrode garment with built-in electrodes that have a non-conductive porous material between the electrode and the user's skin to minimize discomfort to the user's skin.

Also, in United States Patent Application Publication Number US2022/0007750 to Ho, discloses a garment structure for electrotherapy, having positional adjustment of the electrode within the garment and further with a second heating electrode also built in the garment to provide both electrotherapy and heating.

Next, in United States Patent Application Publication Number US2021/0205607 to Herb, discloses a TENS system using phase shifted waveforms with different voltages using an electrode array.

Further, in United States Patent Application Publication Number US2021/0121682 to Mbue, discloses a sock that delivers electrical impulses to the foot and ankle for the treatment of pain using TENS that is wirelessly controlled remotely.

There are numerous patents and applications in the TENS field, which includes not only pain management, but sensory control for bladder control, muscle movement, and feedback from the user either through the electrode or manually for controlling the electrical impulses that the user receives, plus variances in frequency, number of signals, electrode types, and the type of garment for the wearable portion that contains the electrodes being gloves, socks, bands, etc.

The novelty with TENS falls in the following areas; the electrical signal itself (varying frequencies, voltage, and amp levels, multiple signals, different phases, and so on for various therapeutic effects), electrode design and skin attachment (for administering TENS, efficacy, skin irritation issues, and so on), and the garment itself, (glove, sock, wrap, and so on for the position and holding of the electrode).

As noted in TENS literature, a significant issue is with the fact that the electrode needs direct contact with the skin to work properly—i.e. typically adhesively attached, so with gloves, sock, and for pets, would involve not so direct contact with the skin the TENS effectiveness could be compromised, however, this is off set by the benefit of the not so direct contact of the electrode with the user's skin which would reduce the skin irritation that can come with the adhesive from the electrode to the skin attachment,

### SUMMARY OF THE INVENTION

Broadly, the present invention is a sensory control system that is adapted to encompass an outer surface of a particular part of a human body of a user for applying electrical impulses to the outer surface to alter a physical sensation of the particular part of the human body for the user. The sensory control system including a semi planar garment is constructed of a flexible material that is adapted to attach to the outer surface of a particular part of the human body, wherein the semi planar garment is removably engageable to the outer surface of a particular part of the human body, the semi planar garment including a planar garment outer surface, a planar garment inner surface, a planar garment first end portion, and an opposing planar garment second end portion, wherein the semi planar garment substantially assumes a shape of a surrounding sidewall.

The semi planar garment is further constructed of a glove for a human hand that includes a plurality of adjacent surrounding sidewalls forming fourchettes and a thumb that each receive a finger of the human hand such that each said surrounding sidewall has a surrounding sidewall inner surface, wherein the surrounding sidewall is a single layer one piece as between the planar garment outer surface and the planar garment inner surface and the surrounding sidewall inner surface.

A dual bias omnidirectionally movable substantially planar support surface structure assembly that includes a base with a base outer surface and an opposing base inner surface, a support surrounding sidewall with a support surrounding sidewall internal surface and an opposing support surrounding sidewall external surface, the support surrounding sidewall extends from the base about a primary longitudinal axis and the support surrounding sidewall has a support terminating portion wherein the support surrounding sidewall symmetrically arcs with a constant radius arc inward towards the primary longitudinal axis, with the constant radius arc inward having a constant radius arc inside surface and an opposing constant radius arc outside surface, with the support surrounding sidewall terminating portion terminating in an aperture that is disposed about the primary longitudinal axis, the base inner surface, the support surrounding sidewall inner surface, and the support terminating portion constant radius arc inside surface all define a support interior.

A primary spring that is disposed about the primary longitudinal axis, the primary spring having a primary first end portion and an opposing primary second end portion, the primary spring having a spring rate K-one that is in force per unit distance, wherein the primary first end portion is disposed upon the base inner surface, a secondary longitudinal axis that is positioned parallel to the primary longitudinal axis, wherein the secondary longitudinal axis is further positioned in-between the primary longitudinal axis and the support surrounding sidewall internal surface, a secondary spring that is disposed about the secondary longitudinal axis, the secondary spring having a secondary first end portion and an opposing secondary second end portion, the secondary spring having a spring rate K-two that is in force per unit distance that is less than said K-one spring rate, wherein the secondary first end portion is disposed upon the base inner surface, a disc having a disc first end portion and an opposing disc second end portion, the disc second end portion including a button shaped protrusion, the disc is disposed within the support interior positioned such that the disc first end portion is in contact with both the primary spring primary second end portion and the secondary spring secondary second end portion.

A one-half spherical structure having a flat surface with a circular cavity that is sized and configured to freely rotationally receive the button shaped protrusion, the one-half spherical structure has an outer surface that has a constant radius arc that matches the support surrounding sidewall terminating portion constant radius arc inside surface, the one-half spherical structure further has a neck portion extending from the one-half spherical structure constant radius outer surface, wherein the neck portion extends opposite from the flat surface circular cavity along the primary longitudinal axis, the neck portion terminates in the substantially planar support surface that is positioned substantially perpendicular to the primary longitudinal axis.

Wherein positionally the base, the support surrounding sidewall, the support terminating portion, the aperture, the primary spring, the disc, the button shaped protrusion, the circular cavity, the one-half spherical structure, the neck, and the substantially planar support surface are all about the primary longitudinal axis, operationally this results in a default first bias position of the one-half spherical structure constant radius outer surface slidably contacting the constant radius arc inside surface with a portion of the primary longitudinal axis that includes the substantially planar support surface, the neck portion, the one-half spherical structure, the disc, and a portion of the primary spring, and a portion of the secondary spring along the secondary longitudinal axis both to deviate about twenty degrees from vertical from the secondary spring creating an asymmetrical force on the disc from a moment arm distance as between the primary and secondary longitudinal axes resulting in the substantially planar support surface angled in an insert and removal state position, the base outer surface and a portion of the support surrounding sidewall external surface are removably engaged to the inner surface of the surrounding sidewall to mount the dual bias omnidirectionally movable substantially planar support surface structure assembly to be disposed within the glove finger receiving surrounding sidewall.

An electrode that is solely disposed and affixed upon the substantially planar support surface, wherein the electrode is positioned via the insert state position to initially contact a finger tip of the human hand during an insertion of the human hand into the glove to facilitate a low effort insert of the finger to initially slidably contact the electrode at an angle in relation to a tip of the finger, once the finger is fully inserted upon the electrode, the dual bias omnidirectionally movable substantially planar support surface structure assembly moves into an operational position state via creating a second bias that exerts force on the electrode along the primary longitudinal axis away from the base to operationally exert contact force as between the electrode and the finger, even while the finger has omnidirectional movement defined as being vertically, rotationally, tilting front to back, and tilting left to right while the second bias keeps the contact force as between the electrode and the finger, and during operational removal movement of the hand and finger from the glove the dual bias omnidirectionally movable substantially planar support surface structure assembly automatically moves into the default first bias position being ready for a subsequent finger insert into the glove being the plurality of adjacent surrounding sidewalls forming fourchettes and the thumb that each receive the finger of the human hand.

Also included in the sensory control system is control circuitry that is in electrical communication with the electrode, wherein the control circuitry is operative to generate the electrical impulses with particular frequency, phase, pulse versus time amounts, voltage, and ampere levels at user safe low electrical power levels that are less than one-hundred milli-amps and no more than sixty volts.

These and other objects of the present invention will become more readily appreciated and understood from a consideration of the following detailed description of the exemplary embodiments of the present invention when taken together with the accompanying drawings, in which;

### BRIEF DESCRIPTION OF DRAWINGS

FIG. 1 shows a thumb-oriented perspective view of the sensory control system specifically being the semi planar garment in the form of a glove for the human hand;

FIG. 2 shows a palm-oriented perspective view of the sensory control system specifically being the semi planar garment in the form of the glove for the human hand;

FIG. 3 shows section view cut 3-3 from FIGS. 1 and 2, wherein FIG. 3 shows the cross section of a single fingertip encompassed by the finger surrounding sidewall with the dual bias omnidirectionally movable substantially planar support surface structure assembly with electrode shown disposed in contact with the fingertip and to the surrounding sidewall inner surface;

FIG. 4 shows section view cut 4-4 from FIGS. 1 and 2, wherein FIG. 4 shows the cross section of a single fingertip encompassed by the finger surrounding sidewall with the dual bias omnidirectionally movable substantially planar support surface structure assembly with electrode shown disposed in contact with the fingertip and to the surrounding sidewall inner surface, wherein FIG. 4 is rotated ninety degrees to be perpendicular to the section cut view in FIG. 3 for reference;

FIG. 5 shows an upper perspective view of the sensory control system in the form of an orthotic arch support shoe insert sole that includes the dual bias omnidirectionally movable substantially planar support surface structure assembly with the electrode disposed on the foot side first surface;

FIG. 6 shows a use view of an upper perspective view of the sensory control system in the form of an orthotic arch support shoe insert sole with the user's foot in place;

FIG. 7 shows cross section cut 7-7 from FIG. 6, wherein FIG. 7 shows the dual bias omnidirectionally movable substantially planar support surface structure assembly with the electrode disposed between the foot outer surface and the first surface;

FIG. 8 shows cross section cut 8-8 from FIGS. 6 and 7, wherein FIG. 8 shows the dual bias omnidirectionally movable substantially planar support surface structure assembly with the electrode disposed between the foot outer surface and the first surface, wherein FIG. 8 is rotated ninety degrees to be perpendicular to the section cut view in FIG. 7 for reference;

FIG. 9 shows an upper perspective view of the sensory control system in the form of a pet grooming glove that includes the dual bias omnidirectionally movable substantially planar support surface structure assembly with the electrode on the inside exterior surface;

FIG. 10 shows an approaching use upper perspective view of the sensory control system in the form of the pet grooming glove that includes the electrode disposed on the inside exterior surface, wherein the electrode has initial contact with the human head skin surface;

FIG. 11 shows a full contact use upper perspective view of the sensory control system in the form of the pet grooming glove that includes the dual bias omnidirectionally movable substantially planar support surface structure assembly with the electrode disposed on the inside exterior surface, wherein the electrode has full contact with the human head skin surface;

FIG. 12 shows cross section cut 12-12 from FIG. 11, wherein FIG. 12 shows a clearer view of the pet grooming glove that includes the electrode disposed on the inside exterior surface, wherein the dual bias omnidirectionally movable substantially planar support surface structure assembly with the electrode having full contact with the human head skin surface;

FIG. 13 shows cross section cut 13-13 from FIG. 11, wherein FIG. 13 shows a clearer view of the pet grooming glove that includes the dual bias omnidirectionally movable substantially planar support surface structure assembly with the electrode disposed on the inside exterior surface, wherein the electrode has full contact with the human head skin surface, wherein FIG. 13 is rotated ninety degrees to be perpendicular to the section cut view in FIG. 12 for reference;

FIG. 14 shows a side elevation cross section of the dual bias omnidirectionally movable substantially planar support surface structure assembly with electrode that includes the base, the support surrounding sidewall, the terminating portion with the aperture, the primary and secondary springs, the disc, the one-half spherical structure, the neck portion, and the substantially planar support surface;

FIG. 15 shows a side elevation cross section of the dual bias omnidirectionally movable substantially planar support surface structure assembly with the electrode that includes the base, the support surrounding sidewall, the terminating portion with the aperture, the primary and secondary springs, the disc, the one-half spherical structure, the neck portion, and the substantially planar support surface, wherein the dual bias omnidirectionally movable substantially planar support surface structure assembly is in the default first bias position state.

FIG. 16 shows a side elevation cross section of the dual bias omnidirectionally movable substantially planar support surface structure assembly with the electrode that includes the base, the support surrounding sidewall, the terminating portion with the aperture, the primary and secondary springs, the disc, the one-half spherical structure, the neck portion, and the substantially planar support surface, wherein the dual bias omnidirectionally movable substantially planar support surface structure assembly is in the default first bias position state, further shown in the human finger and fingertip having the insertion starting movement with contact onto the electrode that is disposed upon the substantially planar support surface.

FIG. 17 shows a side elevation cross section of the dual bias omnidirectionally movable substantially planar support surface structure assembly with the electrode that includes the base, the support surrounding sidewall, the terminating portion with the aperture, the primary and secondary springs, the disc, the one-half spherical structure, the neck portion, and the substantially planar support surface, wherein the dual bias omnidirectionally movable substantially planar support surface structure assembly is in the second bias operational position state, further shown in the human finger and fingertip having the contact onto the electrode from the force exerted from the second bias operational position state that is disposed upon the substantially planar support surface, noting that the force is substantially consistent with the finger having omnidirectional movement vertically, rotationally, tilting front to back, and tilting left to right.

FIG. 18 shows an exterior side elevation view of the dual bias omnidirectionally movable substantially planar support surface structure assembly with electrode that includes the base, the support surrounding sidewall, the terminating portion with the aperture, the neck portion, and the substantially planar support surface, wherein the dual bias omnidirectionally movable substantially planar support surface structure assembly is in the default first bias position state.

FIG. 19 shows a top view of the dual bias omnidirectionally movable substantially planar support surface structure assembly with electrode that includes the support surrounding sidewall, the terminating portion, and the substantially planar support surface, wherein the dual bias omnidirectionally movable substantially planar support surface structure assembly is in the default first bias position state;

FIG. 20 shows a bottom view of the dual bias omnidirectionally movable substantially planar support surface structure assembly that includes the base, the support surrounding sidewall, and the substantially planar support surface, wherein the dual bias omnidirectionally movable substantially planar support surface structure assembly is in the default first bias position state;

FIG. 21 shows an exterior side elevation view of the dual bias omnidirectionally movable substantially planar support surface structure assembly with electrode that includes the base, the support surrounding sidewall, the terminating portion with the aperture, the neck portion, and the substantially planar support surface, wherein the dual bias omnidirectionally movable substantially planar support surface structure assembly is in the second bias operational position state, also showing the finger and fingertip contacting the electrode utilizing force to keep the contact while accommodating finger movement, including vertical, rotational, tilting front to back, and tilting left to right;

FIG. 22 shows a top view of the dual bias omnidirectionally movable substantially planar support surface structure assembly with electrode (not shown) that includes the support surrounding sidewall, and the substantially planar support surface, wherein the dual bias omnidirectionally movable substantially planar support surface structure assembly is in the second bias operational position state, also showing the finger and fingertip contacting the electrode (not shown) utilizing force (not shown) to keep the contact (not shown) while accommodating finger movement, including vertical, rotational, tilting front to back, and tilting left to right; and

FIG. 23 shows a bottom view of the dual bias omnidirectionally movable substantially planar support surface structure assembly that includes the base, the support surrounding sidewall, wherein the dual bias omnidirectionally movable substantially planar support surface structure assembly is in the second bias operational position state, also showing the finger contacting the electrode (not shown) utilizing force (not shown) to keep the contact (not shown) while accommodating finger movement, including vertical (not shown), rotational, tilting front to back, and tilting left to right.

### DETAILED DESCRIPTION OF THE INVENTION

50 Sensory Control System
55 User
60 Human body
61 Human hand
62 Palm of the human hand 61
63 Back of the human hand 61
65 Particular portion of the human body 60
70 Outer surface of the human body 60
75 Particular part of the human body 60
76 Human foot
77 Arch skin surface of the human foot 76
78 Human head
79 Skin surface of the human head 78
80 Semi planar garment
85 Outer surface of the planar garment 80
90 Inner surface of the planar garment 80
95 First end portion of the planar garment 80
100 Second end portion of the planar garment 80
105 Surrounding sidewall of the planar garment 80
110 Electrode
111 Electrical impulses from the electrode 110
115 Affixed structure of the electrode 110 to the substantially planar support surface 495
120 Non-adhesive contact of the electrode 110 to the outer surface 70 of the particular part 75 of the human body 60
125 Attached position of the semi planar garment 80 to the outer surface 70 of the particular part 75 of the human body 60
130 Control circuitry
135 Electrical communication between the control circuitry 130 and the electrode 110 that can be in the form of wireless or wired
140 Human hand
145 Finger of the human hand 140
146 Fingertip of the human hand 140
150 Glove for the human hand 140
155 Plurality of adjacent surrounding sidewalls or glove 150 fingers
160 Fourchettes of the glove 150 or glove 150 fingers
165 Thumb of the glove 150
170 Inner surface of each of the surrounding sidewalls 155
175 Affixed structure of the electrode 110 being disposed upon the substantially planar support surface 495
180 Non adhesive contact of the electrode 110 upon the fingertip 146 of the human hand 140
185 Orthotic arch support shoe insert insole
190 Human foot side first surface of the orthotic arch support shoe insert insole 185
195 Shoe sole side second surface of the orthotic arch support shoe insert insole 185
200 Affixed structure of the electrode 110 being disposed upon the substantially planar support surface 495
205 Contact of the electrode 110 upon the arch skin surface 77 of the human foot 76
210 Pet grooming type of glove
215 Grooming glove surrounding sidewall
220 Encompass manner of the grooming glove surrounding sidewall 215 about the human hand palm 62 and human hand back 63
225 Exterior surface of the grooming glove surrounding sidewall 215
230 Inside exterior surface of the exterior surface 225
235 Outside exterior surface of the exterior surface 225
240 Adjacent nature of the inside exterior surface 230 to the palm 62 of the hand 61
245 Adjacent nature of the outside exterior surface 235 to the back 63 of the hand 61
250 Affixed structure of the electrode 110 being disposed upon the substantially planar support surface 495
255 Contact of the electrode 110 upon the portion of the human head 78 skin surface 79
300 Dual bias omnidirectionally movable substantially planar support surface structure assembly
305 Base
310 Outer surface of the base 305
315 Inner surface of the base 305
320 Support surrounding sidewall
325 Internal surface of the support surrounding sidewall 320
330 External surface of the support surrounding sidewall 320
335 Primary longitudinal axis
340 Support terminating portion of the support surrounding sidewall 320
345 Symmetric arc of the support terminating portion 340
350 Constant radial arc of the support terminating portion 340
355 Inside surface having the constant radius arc 350
360 Outside surface having the constant radius arc 350
365 Aperture of the support terminating portion 340
370 Support interior
375 Primary spring
380 Primary first end portion of the primary spring 375
385 Primary second end portion of the primary spring 375
390 Disposed nature of the primary first end portion 380 upon the base inner surface
395 Secondary longitudinal axis
400 Parallel position of the secondary longitudinal axis 395 to the primary longitudinal axis 335
405 In-between position of the secondary longitudinal axis 395 as between the primary longitudinal axis 335 and the internal surface 325 of the support surrounding sidewall 320
410 Secondary spring
415 Secondary first end portion of the secondary spring 410
420 Secondary second end portion of the secondary spring 410
425 Disposed nature of the secondary first end portion 415 upon the base inner surface 315
430 Disc
435 First end portion of the disc 430
440 Second end portion of the disc 430
445 Button shaped protrusion of the disc 430
450 Contact of the disc 430 first end portion 435 to the primary spring 375 second end portion 385 and the secondary spring 410 secondary second end portion 420
455 One-half spherical structure
460 Flat surface of the one-half spherical structure 455
465 Circular cavity of the one-half spherical structure 455
470 Freely rotationally receiving of the button shaped protrusion 445 by the circular cavity 465
475 Outer surface of the one-half spherical structure 455
480 Constant radius arc of the outer surface 475
485 Matching of the constant radius arc 480 outer surface 475 and the inside surface 355 having the constant radius arc 350
490 Neck portion of the one-half spherical structure 455
495 Substantially planar support surface
500 Perpendicular position of the substantially planar support surface 495 to the primary longitudinal axis 335
505 Default first bias position also known as the insert and removal state position, also as initial contact state position both being for the planar support surface 495 and the electrode 110
510 Slidable contact of the outer surface 475 to the inside surface 355
515 Moment arm distance as between the primary 335 and secondary 395 longitudinal axes
520 Asymmetrical force of the secondary spring 410
525 Portion of the primary longitudinal axis 335 positioned at about twenty degrees 526 from vertical via the secondary spring 410 asymmetrical force 520 on the disc 430 from the moment arm distance 515 resulting in angling of the substantially planar support surface 495 that defines the default first bias position 505 also known as the insert and removal state position
526 Angle of about twenty degrees
530 Insertion or starting movement of the finger 145 and finger tip 146 into the glove 150 fingers 155, 160, also including the arch skin surface 77 of the human foot 76 and the skin surface 79 of the human head 78
535 Initial slidable contact on the finger 145 tip 146 onto the angled substantially planar support surface 495 supporting the electrode 110 with the planar support surface 495 in the default first bias position 505
540 Fully inserted state of the finger 145 tip 146 into the glove 150 fingers 155, 160 and into the electrode 110
545 Second bias operational position state of the dual bias omnidirectionally movable substantially planar support surface structure assembly 300 via exerting force 550 upon the electrode 110 being along the primary longitudinal axis 335 from the primary 375 and secondary 410 springs
550 Force exerted in the second bias operational position state 545
555 Contact force between the electrode 110 and the finger 145
560 Finger 145, arch skin surface 77, and human head skin surface 78 omnidirectional movement
565 Vertical movement of the finger 145, arch skin surface 77, and human head skin surface 78 omnidirectional movement 560
570 Rotational movement of the finger 145, arch skin surface 77, and human head skin surface 78 omnidirectional movement 560
575 Tilting front to back of the finger 145, arch skin surface 77, and human head skin surface 78 omnidirectional movement 560
580 Tilting left to right of the finger 145, arch skin surface 77, and human head skin surface 78 omnidirectional movement 560
585 Operational removal movement of the finger 145 from the glove 150 fingers 155, 160, also including the arch skin surface 77 of the human foot 76 and the skin surface 79 of the human head 78
590 First recess cavity for the primary spring 375 first end portion 380
595 Partial nesting of the primary spring 375 first end portion 380 in the first recess cavity 590
600 Second recess cavity for the secondary spring 410 first end portion 415
605 Partial nesting of the secondary spring 410 first end portion 415 in the second recess cavity 600
610 Third recess cavity for the primary spring 375 second end portion 385
615 Partial nesting of the primary spring 375 second end portion 385 in the third recess cavity 610
620 Fourth recess cavity for the secondary spring 410 second end portion 420
625 Partial nesting of the secondary spring 410 second end portion 420 in the fourth recess cavity 620
630 Removable engagement of the base 305 outer surface 310 and a portion of the support surrounding sidewall 320 external surface 330 to the inner surface 90, 170, or to the first surface 190, or to the inside exterior surface 230 to mount the dual bias omnidirectionally movable substantially planar support surface structure assembly 300

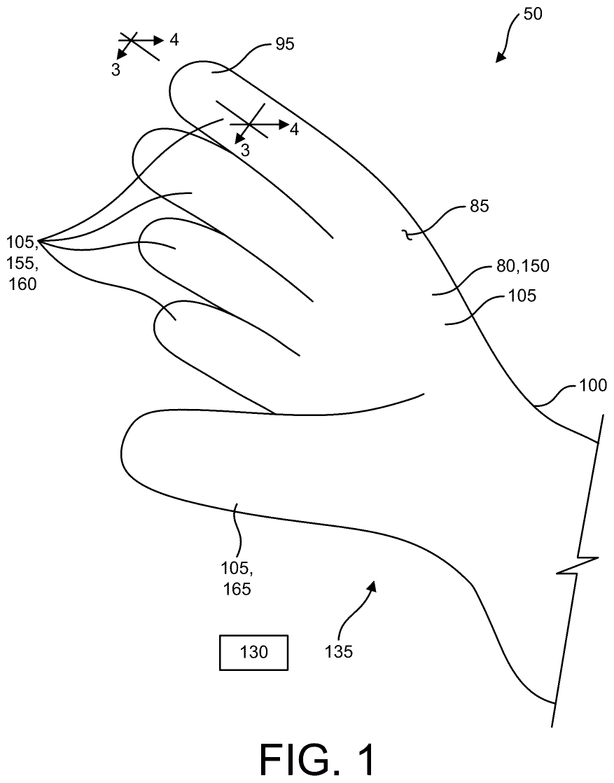

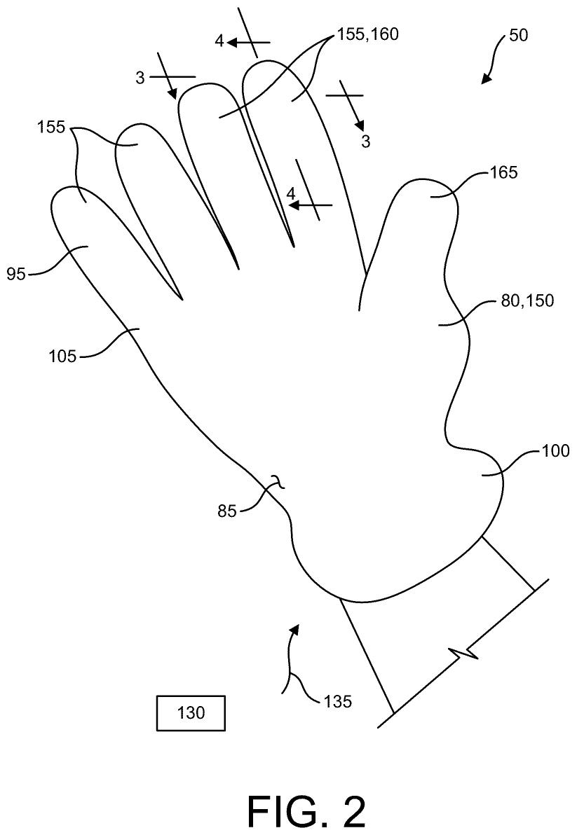

With initial reference to FIG. 1, shown is a thumb-oriented perspective view of the sensory control system 50 specifically being the semi planar garment 80 in the form of a glove for the human hand 150. Next, FIG. 2 shows a palm-oriented perspective view of the sensory control system 50 specifically being the semi planar garment 80 in the form of the glove for the human hand 150.

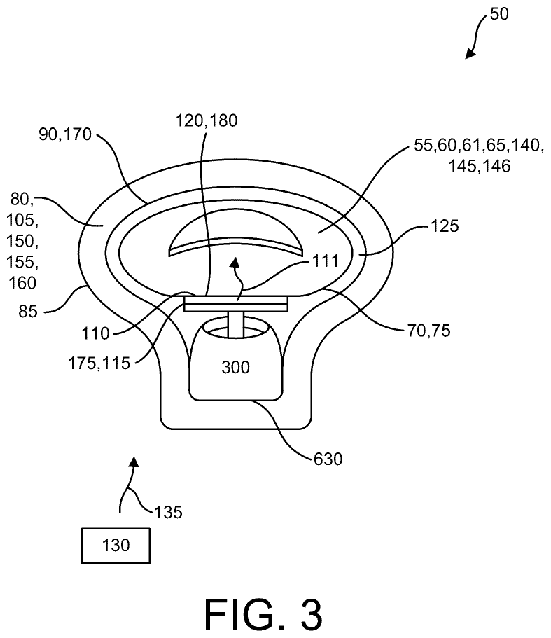

Further, FIG. 3 shows cross section view cut 3-3 from FIGS. 1 and 2, wherein FIG. 3 shows the cross section of a single fingertip 146 encompassed by the finger surrounding sidewall 155 with the dual bias omnidirectionally movable substantially planar support surface structure assembly 300 with the electrode 110 shown disposed in contact with the fingertip 146 and to the surrounding sidewall 155 inner surface 170.

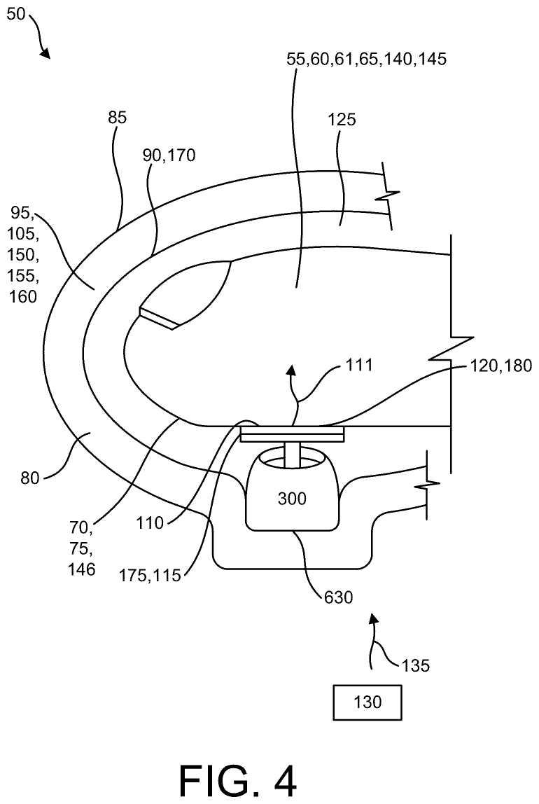

Continuing, FIG. 4 shows cross section view cut 4-4 from FIGS. 1 and 2, wherein FIG. 4 shows the cross section of a single fingertip 146 encompassed by the finger surrounding sidewall 155 the dual bias omnidirectionally movable substantially planar support surface structure assembly 300 with the electrode 110 shown disposed in contact 180 with the fingertip 146 and to the surrounding sidewall 155 inner surface 170, wherein FIG. 4 is rotated ninety degrees to be perpendicular to the cross section cut view in FIG. 3 for reference.

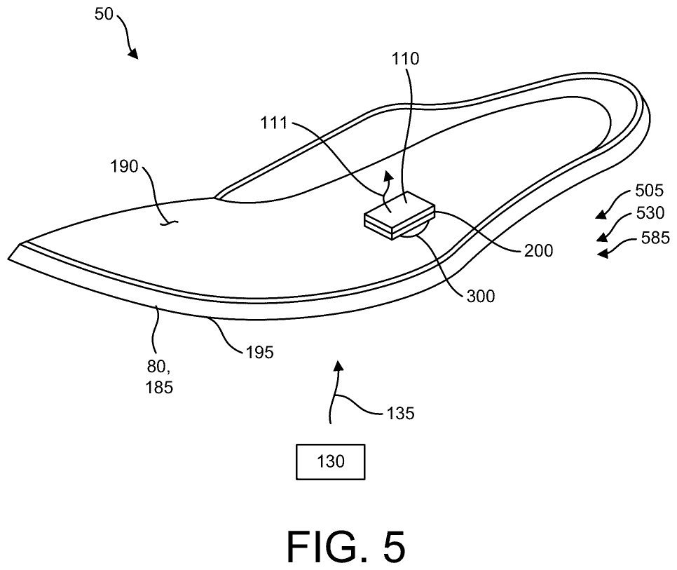

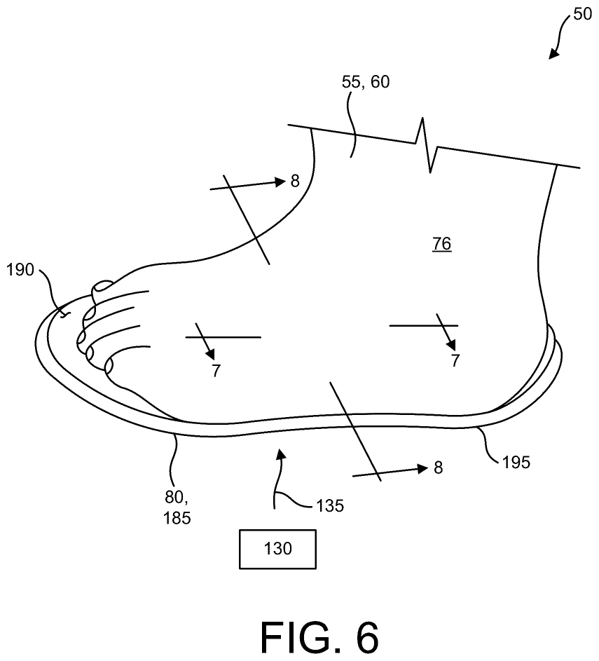

Next, FIG. 5 shows an upper perspective view of the sensory control system 50 in the form of an orthotic arch support shoe insert sole 185 that includes the dual bias omnidirectionally movable substantially planar support surface structure assembly 300 with the electrode 110 disposed on the foot side first surface 190. Also, FIG. 6 shows a use view of an upper perspective view of the sensory control system 50 in the form of an orthotic arch support shoe insert sole 185 with the user's 55 foot 76 in place.

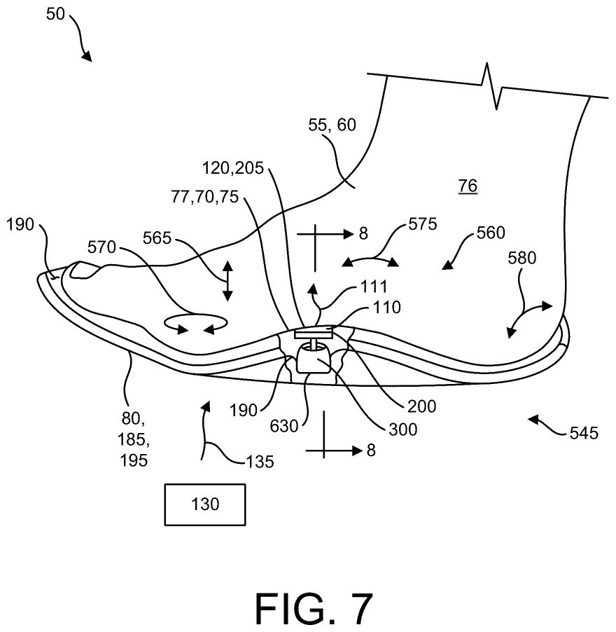

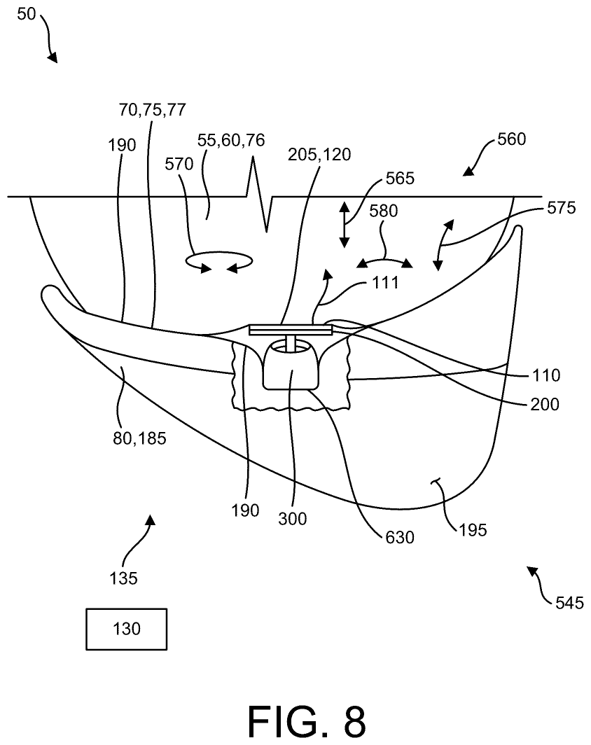

In addition, FIG. 7 shows section cut 7-7 from FIG. 6, wherein FIG. 7 shows the dual bias omnidirectionally movable substantially planar support surface structure assembly 300 with the electrode 110 disposed between the foot outer surface 77 and the first surface 190. Next, FIG. 8 shows section cut 8-8 from FIGS. 6 and 7, wherein FIG. 8 shows the dual bias omnidirectionally movable substantially planar support surface structure assembly 300 with the electrode 110 disposed between the foot outer surface 77 and the first surface 190, wherein FIG. 8 is rotated ninety degrees to be perpendicular to the section cut view in FIG. 7 for reference.

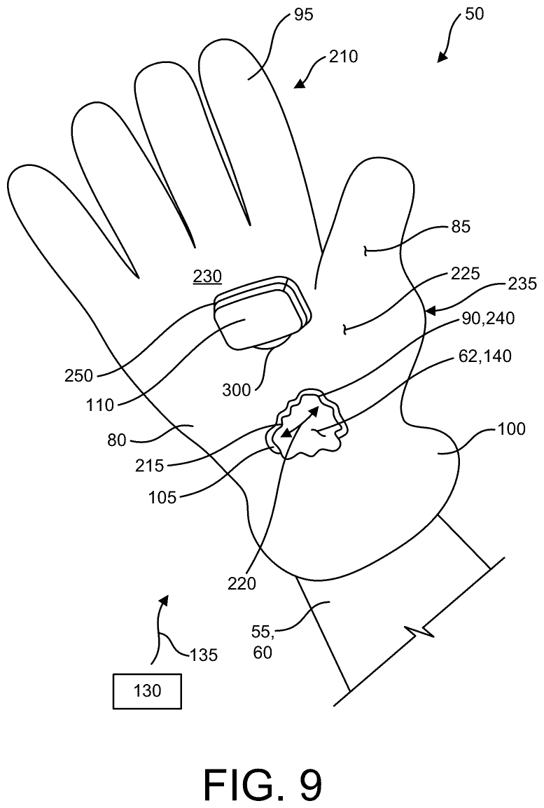

Continuing, FIG. 9 shows an upper perspective view of the sensory control system 50 in the form of a pet grooming glove 210 that includes the dual bias omnidirectionally movable substantially planar support surface structure assembly 300 with the electrode 110 disposed on the inside exterior surface 230.

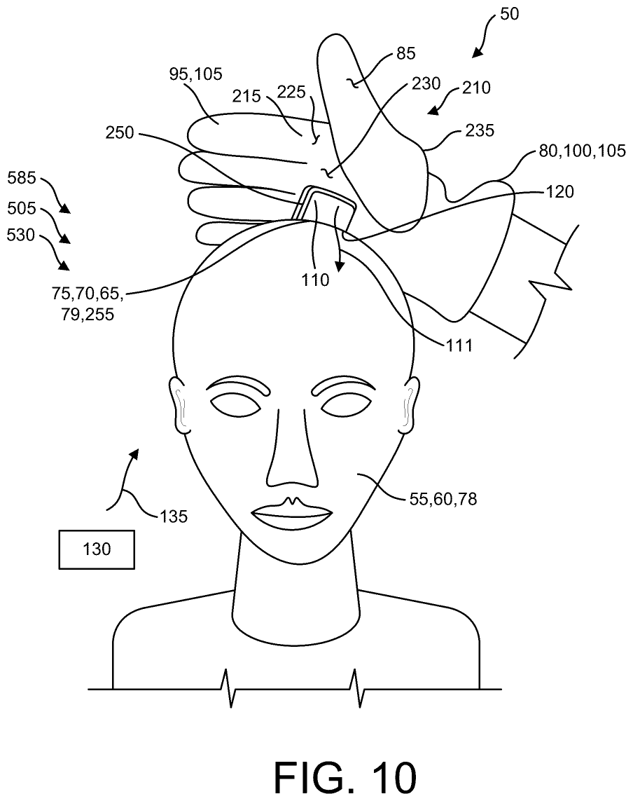

Further, FIG. 10 shows an approaching use upper perspective view of the sensory control system 50 in the form of the pet grooming glove 210 that includes the electrode 110 disposed on the inside exterior surface 230, wherein the electrode 110 has initial contact with the human head 78 skin surface 79.

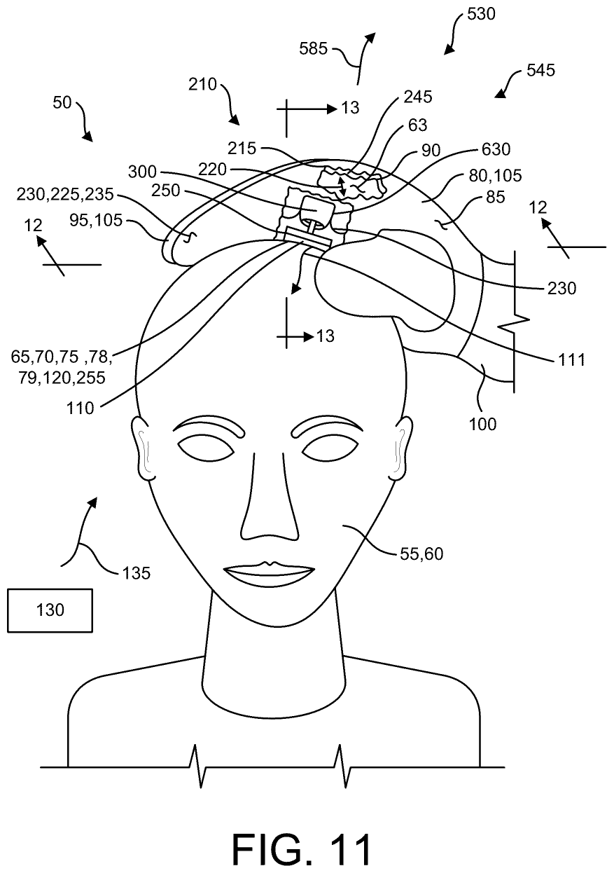

In addition, FIG. 11 shows a full contact use upper perspective view of the sensory control system 50 in the form of the pet grooming glove 210 that includes the dual bias omnidirectionally movable substantially planar support surface structure assembly 300 with the electrode 110 disposed on the inside exterior surface 230, wherein the electrode 110 has full contact with the human head 78 skin surface 79.

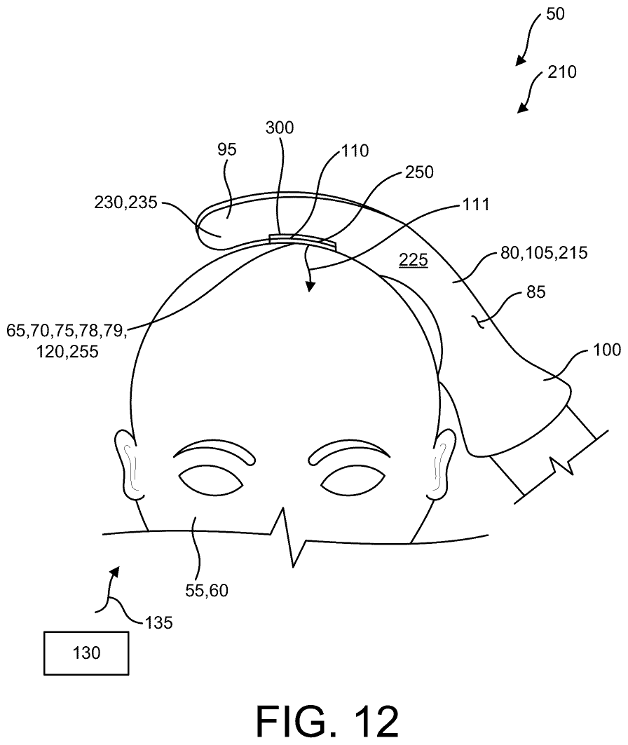

Continuing, FIG. 12 shows cross section cut 12-12 from FIG. 11, wherein FIG. 12 shows a clearer view of the pet grooming glove 210 that includes the dual bias omnidirectionally movable substantially planar support surface structure assembly 300 with the electrode 110 disposed on the inside exterior surface 230, wherein the electrode 110 has full contact with the human head 78 skin surface 79.

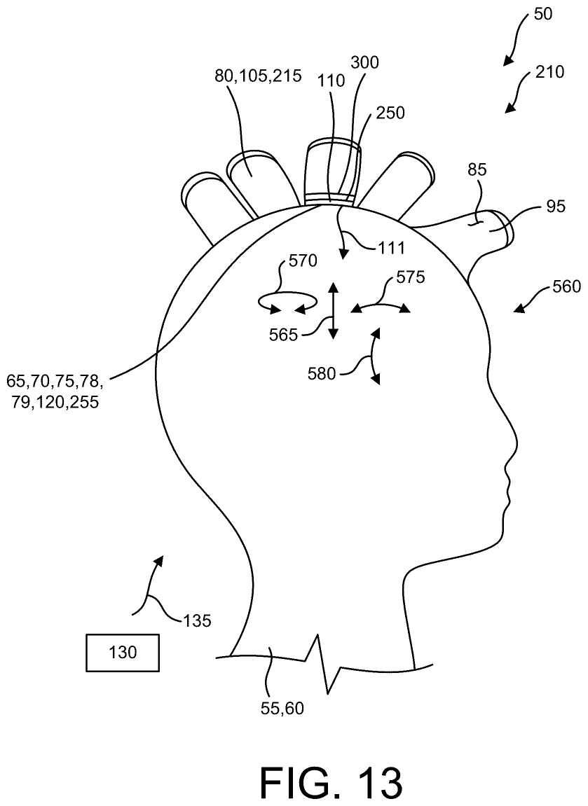

Next, FIG. 13 shows cross section cut 13-13 from FIG. 11, wherein FIG. 13 shows a clearer view of the pet grooming glove 210 that includes the dual bias omnidirectionally movable substantially planar support surface structure assembly 300 with the electrode 110 disposed on the inside exterior surface 230, wherein the electrode 110 has full contact with the human head 78 skin surface 79, wherein FIG. 13 is rotated ninety degrees to be perpendicular to the section cut view in FIG. 12 for reference.

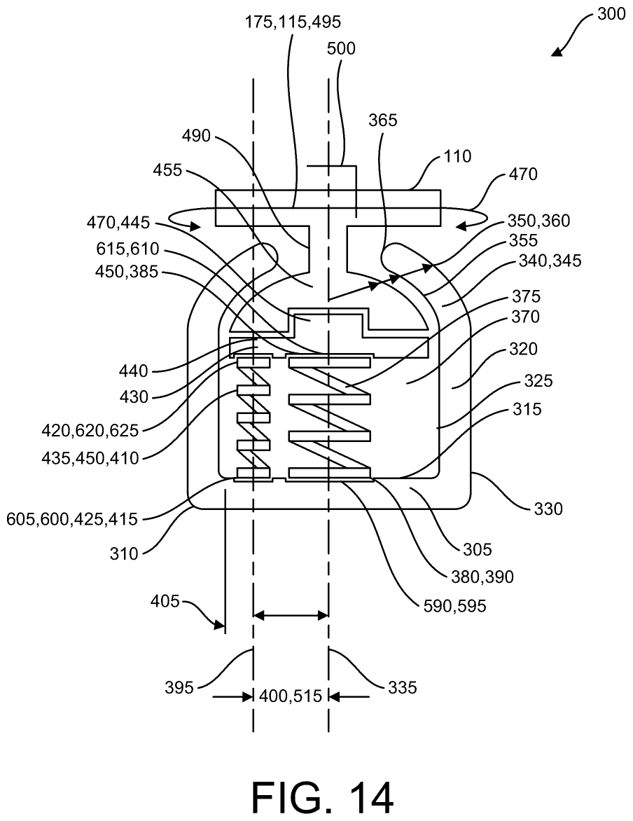

Further, FIG. 14 shows a side elevation cross section of the dual bias omnidirectionally movable substantially planar support surface structure assembly 300 with electrode 110 that includes the base 305, the support surrounding sidewall 320, the terminating portion 340 with the aperture 365, the primary 375 and secondary 410 springs, the disc 430, the one-half spherical structure 455, the neck portion 490, and the substantially planar support surface 495.

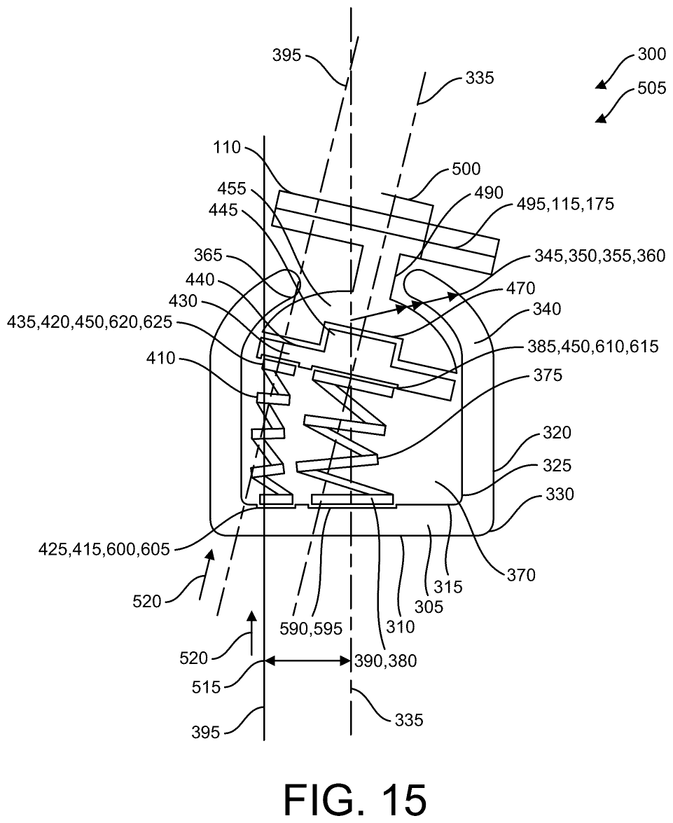

Next, FIG. 15 shows a side elevation cross section of the dual bias omnidirectionally movable substantially planar support surface structure assembly 300 with electrode 110 that includes the base 305, the support surrounding sidewall 320, the terminating portion 340 with the aperture 365, the primary 375 and secondary 410 springs, the disc 430, the one-half spherical structure 455, the neck portion 490, and the substantially planar support surface 495, wherein the dual bias omnidirectionally movable substantially planar support surface structure assembly 300 is in the default first bias position state 505.

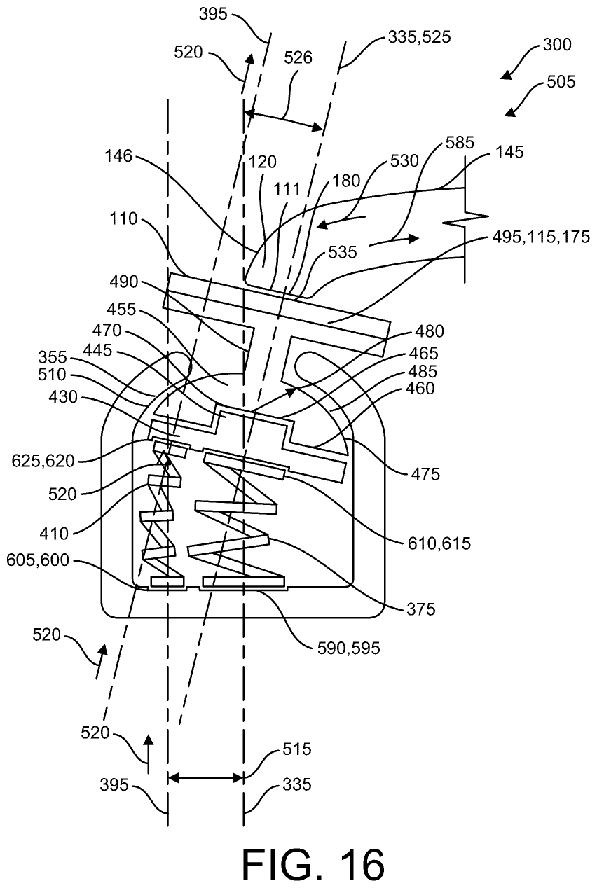

Next, FIG. 16 shows a side elevation cross section of the dual bias omnidirectionally movable substantially planar support surface structure assembly 300 with electrode 110 that includes the base 305, the support surrounding sidewall 320, the terminating portion 340 with the aperture 365, the primary 375 and secondary 410 springs, the disc 430, the one-half spherical structure 455, the neck portion 490, and the substantially planar support surface 495, wherein the dual bias omnidirectionally movable substantially planar support surface structure assembly 300 is in the default first bias position state 505, further shown in the human finger 145 and fingertip 146 having the insertion starting movement 530 with contact 535 onto the electrode 100 that is disposed upon the substantially planar support surface 495.

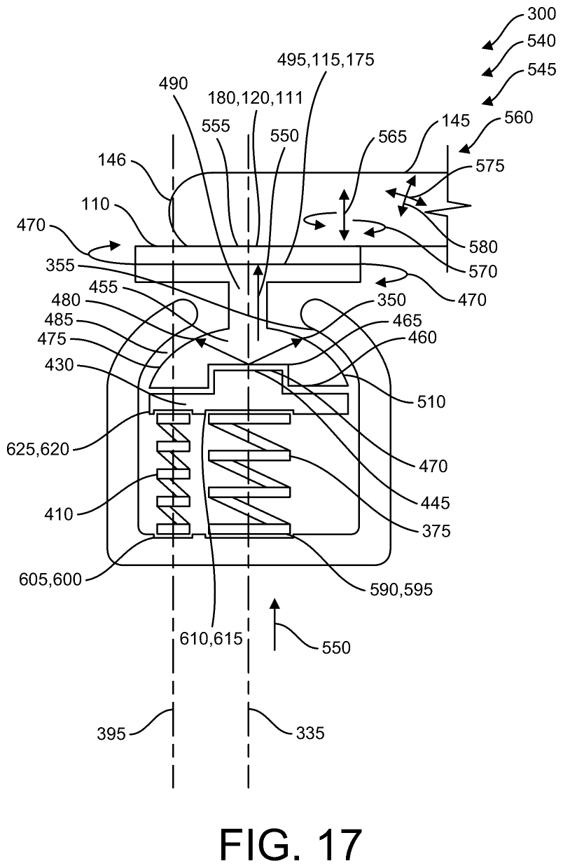

Next, FIG. 17 shows a side elevation cross section of the dual bias omnidirectionally movable substantially planar support surface structure assembly 300 with electrode 110 that includes the base 305, the support surrounding sidewall 320, the terminating portion 340 with the aperture 365, the primary 375 and secondary 410 springs, the disc 430, the one-half spherical structure 455, the neck portion 490, and the substantially planar support surface 495, wherein the dual bias omnidirectionally movable substantially planar support surface structure assembly 300 is in the second bias operational position state 545, further shown in the human finger 145 and fingertip 146 having the contact 535 onto the electrode 110 from the force exerted 550 from the second bias operational position state 545 that is disposed upon the substantially planar support surface 495, noting that the force 550 is substantially consistent with the finger 145 having omnidirectional movement 560 vertically 565, rotationally 570, tilting front to back 575, and tilting left to right 580.

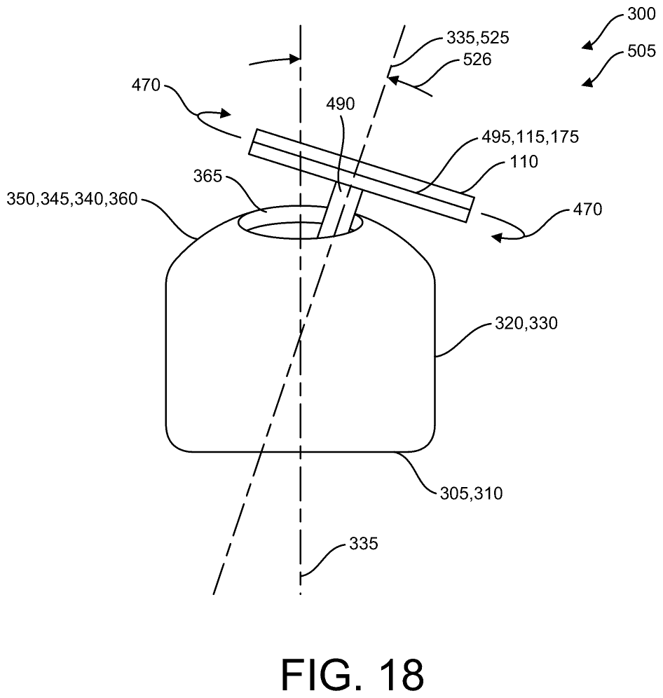

Continuing, FIG. 18 shows an exterior side elevation view of the dual bias omnidirectionally movable substantially planar support surface structure assembly 300 with electrode 110 that includes the base 305, the support surrounding sidewall 320, the terminating portion 340 with the aperture 365, the neck portion 490, and the substantially planar support surface 495, wherein the dual bias omnidirectionally movable substantially planar support surface structure assembly 300 is in the default first bias position state 505.

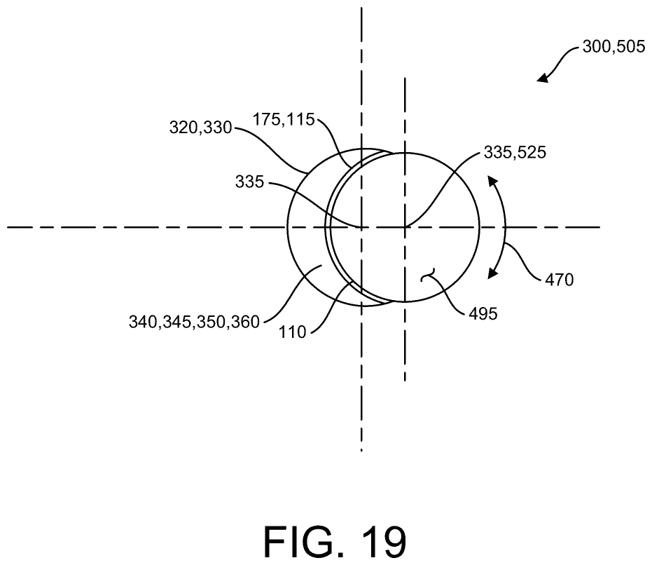

Moving onward, FIG. 19 shows a top view of the dual bias omnidirectionally movable substantially planar support surface structure assembly 300 with the electrode 110 that includes the support surrounding sidewall 320, the terminating portion 340, and the substantially planar support surface 495, wherein the dual bias omnidirectionally movable substantially planar support surface structure assembly 300 is in the default first bias position state 505;

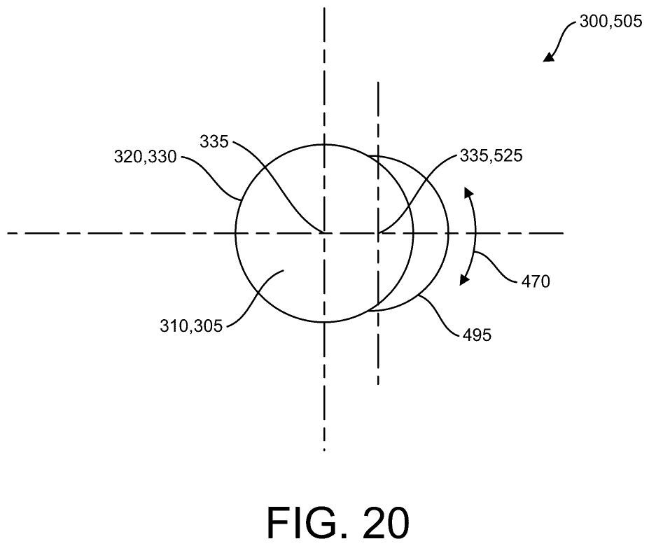

Further, FIG. 20 shows a bottom view of the dual bias omnidirectionally movable substantially planar support surface structure assembly 300 that includes the base 305, the support surrounding sidewall 320, and the substantially planar support surface 495, wherein the dual bias omnidirectionally movable substantially planar support surface structure assembly 300 is in the default first bias position state 505.

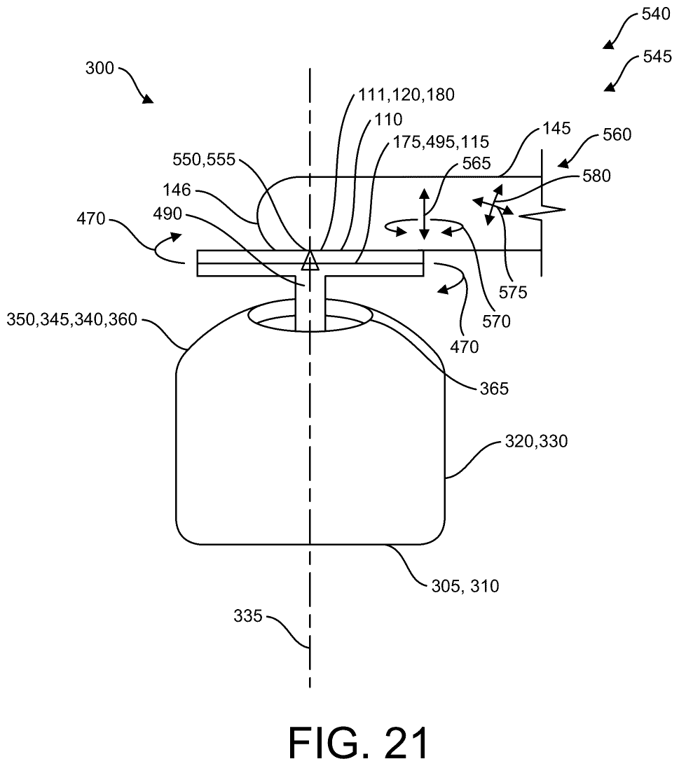

Continuing, FIG. 21 shows an exterior side elevation view of the dual bias omnidirectionally movable substantially planar support surface structure assembly 300 with electrode 110 that includes the base 305, the support surrounding sidewall 320, the terminating portion 340 with the aperture 365, the neck portion 490, and the substantially planar support surface 495, wherein the dual bias omnidirectionally movable substantially planar support surface structure assembly 300 is in the second bias operational position state 545, also showing the finger 145 and fingertip 146 contacting 555 the electrode 110 utilizing force 550 to keep the contact 555 while accommodating finger 145 movement 560, including vertical 565, rotational 570, tilting front to back 575, and tilting left to right 580.

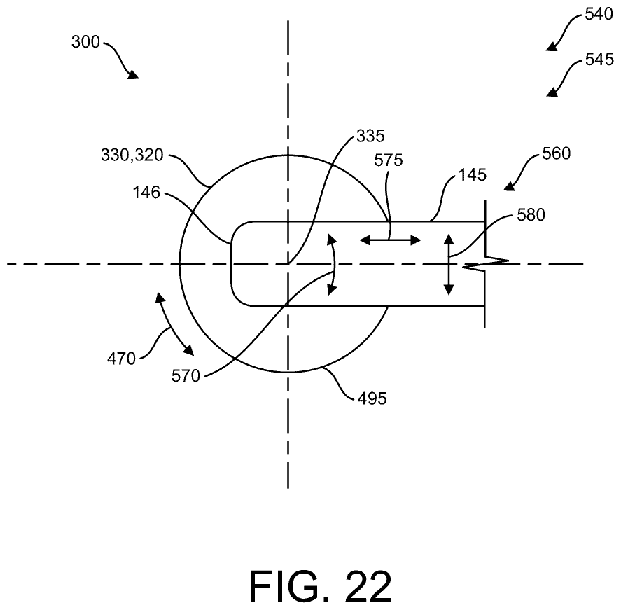

Next, FIG. 22 shows a top view of the dual bias omnidirectionally movable substantially planar support surface structure assembly 300 with electrode 110 that includes the support surrounding sidewall 320, and the substantially planar support surface 495, wherein the dual bias omnidirectionally movable substantially planar support surface structure assembly 300 is in the second bias operational position state 545, also showing the finger 145 and fingertip 146 contacting 555 the electrode 110 (not shown) utilizing force 550 (not shown) to keep the contact 555 (not shown) while accommodating finger 145 movement 560, including vertical 565, rotational 570, tilting front to back 575, and tilting left to right 580.

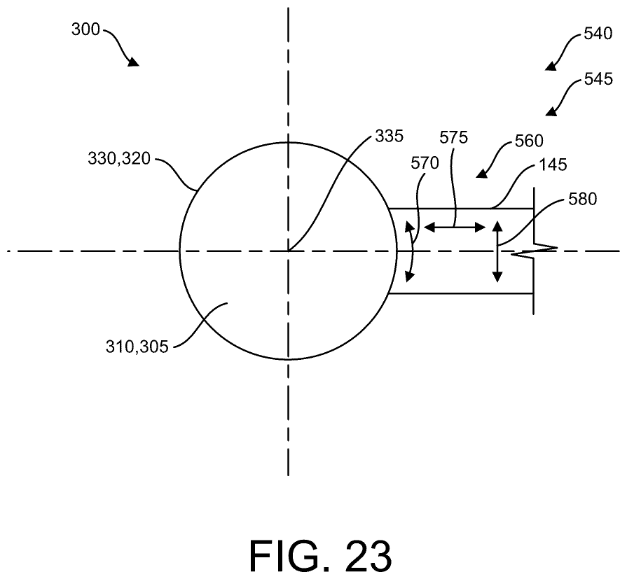

Continuing, FIG. 23 shows a bottom view of the dual bias omnidirectionally movable substantially planar support surface structure assembly 300 that includes the base 305, the support surrounding sidewall 320, wherein the dual bias omnidirectionally movable substantially planar support surface structure assembly 300 is in the second bias operational position state 545, also showing the finger 145 contacting 555 the electrode 110 (not shown) utilizing force 550 (not shown) to keep the contact 555 (not shown) while accommodating finger 145 movement 560, including vertical 565 (not shown), rotational 570, tilting front to back 575, and tilting left to right 580.

Broadly, in looking at FIGS. 1 to 23, the present invention is a sensory control system 50 that is adapted to encompass an outer surface 70 of a particular part 75 or portion 65 of the human body 60 of the user 55 for applying electrical impulses 111 to the outer surface 70 to functionally alter a physical sensation of the particular part 75 of the human body 60 for the user 55.

The sensory control system 50 including a semi planar garment 80 constructed of a flexible material that is adapted to attach to the outer surface 70 of the particular part 75 of the human body 60, wherein the semi planar garment 80 is removably engageable to the outer surface 70 of the particular part 75 of the human body 60, the semi planar garment 80 including a planar garment outer surface 85, a planar garment inner surface 90, a planar garment first end portion 95, and an opposing planar garment second end portion 100, wherein the semi planar garment 80 substantially assumes a shape of a surrounding sidewall 105.

A dual bias omnidirectionally movable substantially planar support surface structure assembly 300 that includes a base 305 with a base outer surface 310 and an opposing base inner surface 315, a support surrounding sidewall 320 with a support surrounding sidewall internal surface 325 and an opposing support surrounding sidewall external surface 330, the support surrounding sidewall 320 extends from the base 305 about a primary longitudinal axis 335 and the support surrounding sidewall 320 has a support terminating portion 340 wherein the support surrounding sidewall 320 symmetrically arcs 345 with a constant radius arc 350 inward towards the primary longitudinal axis 335, with the constant radius arc inward 350 having a constant radius arc inside surface 355 and an opposing constant radius arc outside surface 360, with the support surrounding sidewall terminating portion 340 terminating in an aperture 365 that is disposed about the primary longitudinal axis 335, the base inner surface 315, the support surrounding sidewall internal surface 325, and the support terminating portion 340 constant radius arc inside surface 355 all define a support interior 370, see FIGS. 14 to 23.

A primary spring 375 that is disposed about the primary longitudinal axis 335, the primary spring 375 having a primary first end portion 380 and an opposing primary second end portion 385, the primary spring 375 having a spring rate K-one that is in force per unit distance, wherein the primary first end portion 380 is disposed upon the base inner surface 315, a secondary longitudinal axis 395 that is positioned parallel 400 to the primary longitudinal axis 335, wherein the secondary longitudinal axis 395 is further positioned in-between 405 the primary longitudinal axis 335 and the support surrounding sidewall internal surface 325, a secondary spring 410 that is disposed about the secondary longitudinal axis 395, the secondary spring 410 having a secondary first end portion 415 and an opposing secondary second end portion 420, the secondary spring 410 having a spring rate K-two that is in force per unit distance that is less than said K-one spring rate, wherein the secondary first end portion 415 is disposed upon the base inner surface 315, a disc 430 having a disc first end portion 435 and an opposing disc second end portion 440, the disc second end portion including a button shaped protrusion 445, the disc 430 is disposed within the support interior 370 positioned such that the disc first end portion 435 is in contact 450 with both the primary spring 375 primary second end portion 385 and the secondary spring 410 secondary second end portion 420, see FIGS. 14 to 17.

A one-half spherical structure 455 having a flat surface 460 with a circular cavity 465 that is sized and configured to freely rotationally receive 470 the button shaped protrusion 445, the one-half spherical structure 455 has an outer surface 475 that has a constant radius arc 480 that matches the support surrounding sidewall 320 terminating portion 340 constant radius arc inside surface 355, the one-half spherical structure 455 further has a neck portion 490 extending from the one-half spherical structure 455 constant radius outer surface 480, wherein the neck portion 490 extends opposite from the flat surface circular cavity 465 along the primary longitudinal axis 335, the neck portion 490 terminates in the substantially planar support surface 495 that is positioned substantially perpendicular 500 to the primary longitudinal axis 335, see FIGS. 14 to 17.

Wherein positionally the base 305, the support surrounding sidewall 320, the support terminating portion 340, the aperture 365, the primary spring 375, the disc 430, the button shaped protrusion 445, the circular cavity 465, the one-half spherical structure 455, the neck 490, and the substantially planar support surface 495 are all about the primary longitudinal axis 335, operationally this results in a default first bias position 505 of the one-half spherical structure 455 constant radius outer surface 480 slidably contacting 510 the constant radius arc inside surface 355 with a portion 525 of the primary longitudinal axis 335 that includes the substantially planar support surface 495, the neck portion 490, the one-half spherical structure 455, the disc 430, and a portion of the primary spring 375, and a portion of the secondary spring 410 along the secondary longitudinal axis 395 both to deviate about twenty degrees 525 from vertical from the secondary spring 410 creating an asymmetrical force 520 on the disc 430 from a moment arm distance 515 as between the primary 335 and secondary 395 longitudinal axes resulting in the substantially planar support surface 495 angled in an insert and removal state position 505, the base outer surface 310 and a portion of the support surrounding sidewall 320 external surface 330 are removably engaged 630 to the inner surface 90, 170 of the surrounding sidewall 155 to mount the dual bias omnidirectionally movable substantially planar support surface structure assembly 300 to be disposed within the glove 150 finger receiving surrounding sidewall 155, see in particular FIGS. 3 and 4.

An electrode 110 that is solely disposed and affixed 115, 175 upon the substantially planar support surface 495, wherein the electrode 110 is positioned via the insert state position 505 to initially contact 535 a fingertip 146 of the human hand 61 during an insertion 530 of the human hand 61 into the glove 150 to facilitate a low effort insert of the finger 145 to initially slidably contact 535 the electrode 110 at an angle in relation to a tip 146 of the finger 145, once the finger 145 is fully inserted 540 upon the electrode 110, the dual bias omnidirectionally movable substantially planar support surface structure assembly 300 moves into an operational position state 545 via creating a second bias that exerts force 550 on the electrode 110 along the primary longitudinal axis 335 away from the base 305 to operationally exert contact force 555 as between the electrode 110 and the finger 145, even while the finger 145 has omnidirectional movement 560 defined as being vertically 565, rotationally 570, tilting front to back 575, and tilting left to right 580 while the second bias 545 keeps the contact force 555 as between the electrode 110 and the finger 145, and during operational removal movement 585 of the hand 61 and finger 145 from the glove 150 the dual bias omnidirectionally movable substantially planar support surface structure assembly 300 automatically moves into the default first bias position 505 being ready for a subsequent finger 145 insert 530 into the glove 150 being the plurality of adjacent surrounding sidewalls 155 forming fourchettes 160 and the thumb 165 that each receive the finger 145 of the human hand 61, see FIGS. 3, 4, 16, and 17.

Also included in the sensory control system 50 is control circuitry 130 that is in electrical communication 135 with the electrode 110, wherein the control circuitry 130 is operative to generate the electrical impulses 111 with particular frequency, phase, pulse versus time amounts, voltage, and ampere levels at user 55 safe low electrical power levels that are less than one-hundred milli-amps and no more than sixty volts.

As an option for the sensory control system 50, wherein the electrode 110 and the control circuitry 130 can be constructed of a transcutaneous electrical nerve stimulation (TENS) system.

The semi planar garment 80 can be constructed of a glove 150 for a human hand 61 that includes a plurality of adjacent surrounding sidewalls 155 forming fourchettes 160 and a thumb 165 that each receive a finger 145 of the human hand 61 such that each surrounding sidewall 155 has a surrounding sidewall inner surface 170, see FIGS. 1 to 4.

Alternatively, for the sensory control system 50, wherein the semi planar garment 80 is constructed of an orthotic arch support shoe insert insole 185 that includes a human foot side first surface 190 and an opposing shoe sole side second surface 195, see FIGS. 5 to 8.

A further alternative for the sensory control system 50, wherein the semi planar garment 80 is constructed of a pet grooming type of glove 210 for a human hand 61 that includes a grooming glove 210 surrounding sidewall 215 that is positioned to encompass 220 a human hand 61 palm 62 and a human hand 61 back of the hand 63, the grooming glove surrounding sidewall 215 including an exterior surface 225 that is divided into an inside exterior surface 230 being adjacent 240 to the palm 62 of the hand 61 and an outside exterior surface 235 being adjacent 245 to the back 63 of the hand 61, see FIGS. 9 to 13.

Optionally for the sensory control system 50, wherein for the dual bias omnidirectionally movable substantially planar support surface structure assembly 300, the base 305 inner surface 315 further comprises a primary spring 375 first end portion 380 first recess cavity 590 that partially nests 595 the primary spring 375 first end portion 380 to operationally help keep the primary spring 375 first end portion 380 in position from having movement away from being about the primary longitudinal axis 335 and the base 305 inner surface 315 further comprises a secondary spring 410 first end portion 415 second recess cavity 600 that partially nests 605 the secondary spring 410 first end portion 415 to operationally help keep the secondary spring 410 first end portion 415 in position from having movement away from being about the secondary longitudinal axis 395, see FIGS. 14 to 17.

Optionally for the sensory control system 50, wherein for the dual bias omnidirectionally movable substantially planar support surface structure assembly 300, the disc 430 first end portion 435 further comprises a primary spring 375 second end portion 385 third recess cavity 610 that partially nests 615 the primary spring 375 second end portion 385 to operationally help keep the primary spring 375 second end portion 385 in position from having movement away from being about the primary longitudinal axis 335 and the disc 430 first end portion 435 further comprises a secondary spring 410 second end portion 420 a fourth recess cavity 620 that partially nests 625 the secondary spring 410 second end portion 420 to operationally help keep the secondary spring 410 second end portion 420 in position from having movement away from being about the secondary longitudinal axis 395, see FIGS. 14 to 17.

The base 305 outer surface 310 and a portion of the support surrounding sidewall 320 external surface 330 are removably engaged 630 to the inside exterior surface 230 to mount the dual bias omnidirectionally movable substantially planar support surface structure assembly 300 in the sidewall 215 of the pet grooming type of glove 210, see FIG. 11.

An electrode 110 that is solely disposed and affixed upon the substantially planar support surface 495, wherein the electrode 110 is positioned via the default first bias position initial contact state position 505 to initially contact 120, 255 a portion of a human head 78 skin surface 79 to facilitate a soft contact of the portion of the human head 78 skin surface 79 after which the dual bias omnidirectionally movable substantially planar support surface structure assembly 300 moves into an operational position state 545 via creating a second bias that exerts force 550 on the electrode 110 along the primary longitudinal axis 335 away from the base 305 to operationally exert contact force 255 as between the electrode 110 and the portion of the human head 78 skin surface 79, even while the portion of the human head 78 skin surface 79 has omnidirectional movement 560 defined as being vertically 565, rotationally 570, tilting front to back 575, and tilting left to right 580 while the second bias 545 keeps the contact force 550 as between the electrode 110 and the portion of the human head 78 skin surface 79, and during operational removal movement 585 of the pet grooming type of glove 210 from the portion of the human head 78 skin surface 79, the dual bias omnidirectionally movable substantially planar support surface structure assembly 300 automatically moves into the default first bias position initial contact state position 505 being ready for a subsequent contact of the portion of the human head 78 skin surface 79, see FIGS. 9 to 13.

The base 305 outer surface 310 and a portion of the support surrounding sidewall 320 external surface 330 are removably engaged 630 to the human foot 76 side first surface 190 to mount the dual bias omnidirectionally movable substantially planar support surface structure assembly 300 in the orthotic arch support shoe insert insole 185, see FIGS. 5 to 8.

An electrode 110 that is solely disposed and affixed upon the substantially planar support surface 495, wherein the electrode 110 is positioned via the default first bias position initial contact state position 505 to initially contact 120, 205 a portion of a human foot 76 arch skin surface 77 to facilitate a soft contact of the portion of the human foot 76 arch skin surface 77 after which the dual bias omnidirectionally movable substantially planar support surface structure assembly 300 moves into an operational position state 545 via creating a second bias that exerts force 550 on the electrode 110 along the primary longitudinal axis 335 away from the base 305 to operationally exert contact force 550, 205 as between the electrode 110 and the portion of the human foot 76 arch skin surface 77, even while the portion of the human foot 76 arch skin surface 77 has omnidirectional movement 560 defined as being vertically 565, rotationally 570, tilting front to back 575, and tilting left to right 580 while the second bias 545 keeps the contact force 550, 205 as between the electrode 110 and the portion of the human foot 76 arch skin surface 77, and during operational removal movement 585 of the human foot 76 side first surface 190 from the portion of the human foot 76 arch skin surface 77, the dual bias omnidirectionally movable substantially planar support surface structure assembly 300 automatically moves into the default first bias position initial contact state position 505 being ready for a subsequent contact of the portion of the human foot 76 arch skin surface 77, see FIGS. 5 to 8.

Accordingly, the present invention of a Sensory Control System has been described with some degree of particularity directed to the embodiments of the present invention. It should be appreciated, though; that the present invention is defined by the following claims construed in light of the prior art so modifications or changes may be made to the exemplary embodiments of the present invention without departing from the inventive concepts contained therein.

1. A sensory control system that is adapted to encompass an outer surface of a particular part of a human body of a user for applying electrical impulses to the outer surface to alter a physical sensation of the particular part of the human body for the user, said sensory control system comprising:
a. a semi planar garment constructed of a flexible material that is adapted to attach to the outer surface of a particular part of the human body, wherein said semi planar garment is removably engageable to the outer surface of a particular part of the human body, said semi planar garment including a planar garment outer surface, a planar garment inner surface, a planar garment first end portion, and an opposing planar garment second end portion, wherein said semi planar garment substantially assumes a shape of a surrounding sidewall, said semi planar garment is further constructed of a glove for a human hand that includes a plurality of adjacent surrounding sidewalls forming fourchettes and a thumb that each receive a finger of the human hand such that each said surrounding sidewall has a surrounding sidewall inner surface, wherein said surrounding sidewall is a single layer one piece as between said planar garment outer surface and said planar garment inner surface and said surrounding sidewall inner surface;
b. a dual bias omnidirectionally movable substantially planar support surface structure assembly that includes a base with a base outer surface and an opposing base inner surface, a support surrounding sidewall with a support surrounding sidewall internal surface and an opposing support surrounding sidewall external surface, said support surrounding sidewall extends from said base about a primary longitudinal axis and said support surrounding sidewall has a support terminating portion wherein said support surrounding sidewall symmetrically arcs with a constant radius arc inward towards said primary longitudinal axis, with said constant radius arc inward having a constant radius arc inside surface and an opposing constant radius arc outside surface, with said support surrounding sidewall terminating portion terminating in an aperture that is disposed about said primary longitudinal axis, said base inner surface, said support surrounding sidewall internal surface, and said support terminating portion constant radius arc inside surface all define a support interior, a primary spring that is disposed about said primary longitudinal axis, said primary spring having a primary first end portion and an opposing primary second end portion, said primary spring having a spring rate K-one that is in force per unit distance, wherein said primary first end portion is disposed upon said base inner surface, a secondary longitudinal axis that is positioned parallel to said primary longitudinal axis, wherein said secondary longitudinal axis is further positioned in-between said primary longitudinal axis and said support surrounding sidewall internal surface, a secondary spring that is disposed about said secondary longitudinal axis, said secondary spring having a secondary first end portion and an opposing secondary second end portion, said secondary spring having a spring rate K-two that is in force per unit distance that is less than said K-one spring rate, wherein said secondary first end portion is disposed upon said base inner surface, a disc having a disc first end portion and an opposing disc second end portion, said disc second end portion including a button shaped protrusion, said disc is disposed within said support interior positioned such that said disc first end portion is in contact with both said primary spring primary second end portion and said secondary spring secondary second end portion, a one-half spherical structure having a flat surface with a circular cavity that is sized and configured to freely rotationally receive said button shaped protrusion, said one-half spherical structure has an outer surface that has a constant radius arc that matches said support surrounding sidewall terminating portion constant radius arc inside surface, said one-half spherical structure further has a neck portion extending from said one-half spherical structure constant radius outer surface, wherein said neck portion extends opposite from said flat surface circular cavity along said primary longitudinal axis, said neck portion terminates in said substantially planar support surface that is positioned substantially perpendicular to said primary longitudinal axis, wherein positionally said base, said support surrounding sidewall, said support terminating portion, said aperture, said primary spring, said disc, said button shaped protrusion, said circular cavity, said one-half spherical structure, said neck, and said substantially planar support surface are all about said primary longitudinal axis, operationally this results in a default first bias position of said one-half spherical structure constant radius outer surface slidably contacting said constant radius arc inside surface with a portion of said primary longitudinal axis that includes said substantially planar support surface, said neck portion, said one-half spherical structure, said disc, and a portion of said primary spring, and a portion of said secondary spring along said secondary longitudinal axis both to deviate about twenty degrees from vertical from said secondary spring creating an asymmetrical force on said disc from a moment arm distance as between said primary and secondary longitudinal axes resulting in said substantially planar support surface angled in an insert and removal state position, said base outer surface and a portion of said support surrounding sidewall external surface are removably engaged to said inner surface of said surrounding sidewall to mount said dual bias omnidirectionally movable substantially planar support surface structure assembly to be disposed within said glove finger receiving surrounding sidewall;
c. an electrode that is solely disposed and affixed upon said substantially planar support surface, wherein said electrode is positioned via said insert state position to initially contact a finger tip of the human hand during an insertion of the human hand into said glove to facilitate a low effort insert of the finger to initially slidably contact said electrode at an angle in relation to a tip of the finger, once the finger is fully inserted upon said electrode, said dual bias omnidirectionally movable substantially planar support surface structure assembly moves into an operational position state via creating a second bias that exerts force on said electrode along said primary longitudinal axis away from said base to operationally exert contact force as between said electrode and the finger, even while the finger has omnidirectional movement defined as being vertically, rotationally, tilting front to back, and tilting left to right while said second bias keeps the contact force as between said electrode and the finger, and during operational removal movement of the hand and finger from said glove said dual bias omnidirectionally movable substantially planar support surface structure assembly automatically moves into said default first bias position being ready for a subsequent finger insert into said glove being said plurality of adjacent surrounding sidewalls forming fourchettes and said thumb that each receive the finger of the human hand; and
d. control circuitry that is in electrical communication with said electrode, wherein said control circuitry is operative to generate the electrical impulses with particular frequency, phase, pulse versus time amounts, voltage, and ampere levels at user safe low electrical power levels that are less than one-hundred milli-amps and no more than sixty volts.

2. A sensory control system according to claim 1 wherein said electrode and said control circuitry are constructed of a transcutaneous electrical nerve stimulation (TENS) system.

3. A sensory control system according to claim 1 wherein said base inner surface further comprises a primary spring first end portion first recess cavity that partially nests said primary spring first end portion to operationally help keep said primary spring first end portion in position about said primary longitudinal axis and said base inner surface further comprises a secondary spring first end portion second recess cavity that partially nests said secondary spring first end portion to operationally help keep said secondary spring first end portion in position about said secondary longitudinal axis.

4. A sensory control system according to claim 1 wherein said disc first end portion further comprises a primary spring second end portion third recess cavity that partially nests said primary spring second end portion to operationally help keep said primary spring second end portion in position about said primary longitudinal axis and said disc first end portion further comprises a secondary spring second end portion fourth recess cavity that partially nests said secondary spring second end portion to operationally help keep said secondary spring second end portion in position about said secondary longitudinal axis.

5. A sensory control system that is adapted to encompass an outer surface of a particular part of a human body of a user for applying electrical impulses to the outer surface to alter a physical sensation of the particular part of the human body for the user, said sensory control system comprising:
a. a pet grooming type of glove for a human hand that includes a grooming glove surrounding sidewall that is positioned to encompass a human hand palm and a human hand back of the hand, said grooming glove surrounding sidewall including an exterior surface that is divided into an inside exterior surface being adjacent to the palm of the hand and an outside exterior surface being adjacent to the back of the hand;
b. a dual bias omnidirectionally movable substantially planar support surface structure assembly that includes a base with a base outer surface and an opposing base inner surface, a support surrounding sidewall with a support surrounding sidewall internal surface and an opposing support surrounding sidewall external surface, said support surrounding sidewall extends from said base about a primary longitudinal axis and said support surrounding sidewall has a support terminating portion wherein said support surrounding sidewall symmetrically arcs with a constant radius arc inward towards said primary longitudinal axis, with said constant radius arc inward having a constant radius arc inside surface and an opposing constant radius arc outside surface, with said support surrounding sidewall terminating portion terminating in an aperture that is disposed about said primary longitudinal axis, said base inner surface, said support surrounding sidewall internal surface, and said support terminating portion constant radius arc inside surface all define a support interior, a primary spring that is disposed about said primary longitudinal axis, said primary spring having a primary first end portion and an opposing primary second end portion, said primary spring having a spring rate K-one that is in force per unit distance, wherein said primary first end portion is disposed upon said base inner surface, a secondary longitudinal axis that is positioned parallel to said primary longitudinal axis, wherein said secondary longitudinal axis is further positioned in-between said primary longitudinal axis and said support surrounding sidewall internal surface, a secondary spring that is disposed about said secondary longitudinal axis, said secondary spring having a secondary first end portion and an opposing secondary second end portion, said secondary spring having a spring rate K-two that is in force per unit distance that is less than said K-one spring rate, wherein said secondary first end portion is disposed upon said base inner surface, a disc having a disc first end portion and an opposing disc second end portion, said disc second end portion including a button shaped protrusion, said disc is disposed within said support interior positioned such that said disc first end portion is in contact with both said primary spring primary second end portion and said secondary spring secondary second end portion, a one-half spherical structure having a flat surface with a circular cavity that is sized and configured to freely rotationally receive said button shaped protrusion, said one-half spherical structure has an outer surface that has a constant radius arc that matches said support surrounding sidewall terminating portion constant radius arc inside surface, said one-half spherical structure further has a neck portion extending from said one-half spherical structure constant radius outer surface, wherein said neck portion extends opposite from said flat surface circular cavity along said primary longitudinal axis, said neck portion terminates in said substantially planar support surface that is positioned substantially perpendicular to said primary longitudinal axis, wherein positionally said base, said support surrounding sidewall, said support terminating portion, said aperture, said primary spring, said disc, said button shaped protrusion, said circular cavity, said one-half spherical structure, said neck, and said substantially planar support surface are all about said primary longitudinal axis, operationally this results in a default first bias position of said one-half spherical structure constant radius outer surface slidably contacting said constant radius arc inside surface with a portion of said primary longitudinal axis that includes said substantially planar support surface, said neck portion, said one-half spherical structure, said disc, and a portion of said primary spring, and a portion of said secondary spring along said secondary longitudinal axis both to deviate about twenty degrees from vertical from said secondary spring creating an asymmetrical force on said disc from a moment arm distance as between said primary and secondary longitudinal axes resulting in said substantially planar support surface angled in said default first bias position with an initial contact state position, said base outer surface and a portion of said support surrounding sidewall external surface are removably engaged to said inside exterior surface to mount said dual bias omnidirectionally movable substantially planar support surface structure assembly in said pet grooming type of glove;
c. an electrode that is solely disposed and affixed upon said substantially planar support surface, wherein said electrode is positioned via said default first bias position initial contact state position to initially contact a portion of a human head skin surface to facilitate a soft contact of the portion of the human head skin surface after which said dual bias omnidirectionally movable substantially planar support surface structure assembly moves into an operational position state via creating a second bias that exerts force on said electrode along said primary longitudinal axis away from said base to operationally exert contact force as between said electrode and the portion of the human head skin surface, even while the portion of the human head skin surface has omnidirectional movement defined as being vertically, rotationally, tilting front to back, and tilting left to right while said second bias keeps the contact force as between said electrode and the portion of the human head skin surface, and during operational removal movement of said pet grooming type of glove from the portion of the human head skin surface, said dual bias omnidirectionally movable substantially planar support surface structure assembly automatically moves into said default first bias position initial contact state position being ready for a subsequent contact of the portion of the human head skin surface; and
d. control circuitry that is in electrical communication with said electrode, wherein said control circuitry is operative to generate the electrical impulses with particular frequency, phase, pulse versus time amounts, voltage, and ampere levels at user safe low electrical power levels that are less than one-hundred milli-amps and no more than sixty volts.

6. A sensory control system according to claim 5 wherein said electrode and said control circuitry are constructed of a transcutaneous electrical nerve stimulation (TENS) system.

7. A sensory control system according to claim 5 wherein said base inner surface further comprises a primary spring first end portion first recess cavity that partially nests said primary spring first end portion to operationally help keep said primary spring first end portion in position about said primary longitudinal axis and said base inner surface further comprises a secondary spring first end portion second recess cavity that partially nests said secondary spring first end portion to operationally help keep said secondary spring first end portion in position about said secondary longitudinal axis.

8. A sensory control system according to claim 5 wherein said disc first end portion further comprises a primary spring second end portion third recess cavity that partially nests said primary spring second end portion to operationally help keep said primary spring second end portion in position about said primary longitudinal axis and said disc first end portion further comprises a secondary spring second end portion fourth recess cavity that partially nests said secondary spring second end portion to operationally help keep said secondary spring second end portion in position about said secondary longitudinal axis.

9. A sensory control system that is adapted to encompass an outer surface of a particular part of a human body of a user for applying electrical impulses to the outer surface to alter a physical sensation of the particular part of the human body for the user, said sensory control system comprising:
a. an orthotic arch support shoe insert insole that includes a human foot side first surface and an opposing shoe sole side second surface;
b. a dual bias omnidirectionally movable substantially planar support surface structure assembly that includes a base with a base outer surface and an opposing base inner surface, a support surrounding sidewall with a support surrounding sidewall internal surface and an opposing support surrounding sidewall external surface, said support surrounding sidewall extends from said base about a primary longitudinal axis and said support surrounding sidewall has a support terminating portion wherein said support surrounding sidewall symmetrically arcs with a constant radius arc inward towards said primary longitudinal axis, with said constant radius arc inward having a constant radius arc inside surface and an opposing constant radius arc outside surface, with said support surrounding sidewall terminating portion terminating in an aperture that is disposed about said primary longitudinal axis, said base inner surface, said support surrounding sidewall internal surface, and said support terminating portion constant radius arc inside surface all define a support interior, a primary spring that is disposed about said primary longitudinal axis, said primary spring having a primary first end portion and an opposing primary second end portion, said primary spring having a spring rate K-one that is in force per unit distance, wherein said primary first end portion is disposed upon said base inner surface, a secondary longitudinal axis that is positioned parallel to said primary longitudinal axis, wherein said secondary longitudinal axis is further positioned in-between said primary longitudinal axis and said support surrounding sidewall internal surface, a secondary spring that is disposed about said secondary longitudinal axis, said secondary spring having a secondary first end portion and an opposing secondary second end portion, said secondary spring having a spring rate K-two that is in force per unit distance that is less than said K-one spring rate, wherein said secondary first end portion is disposed upon said base inner surface, a disc having a disc first end portion and an opposing disc second end portion, said disc second end portion including a button shaped protrusion, said disc is disposed within said support interior positioned such that said disc first end portion is in contact with both said primary spring primary second end portion and said secondary spring secondary second end portion, a one-half spherical structure having a flat surface with a circular cavity that is sized and configured to freely rotationally receive said button shaped protrusion, said one-half spherical structure has an outer surface that has a constant radius arc that matches said support surrounding sidewall terminating portion constant radius arc inside surface, said one-half spherical structure further has a neck portion extending from said one-half spherical structure constant radius outer surface, wherein said neck portion extends opposite from said flat surface circular cavity along said primary longitudinal axis, said neck portion terminates in said substantially planar support surface that is positioned substantially perpendicular to said primary longitudinal axis, wherein positionally said base, said support surrounding sidewall, said support terminating portion, said aperture, said primary spring, said disc, said button shaped protrusion, said circular cavity, said one-half spherical structure, said neck, and said substantially planar support surface are all about said primary longitudinal axis, operationally this results in a default first bias position of said one-half spherical structure constant radius outer surface slidably contacting said constant radius arc inside surface with a portion of said primary longitudinal axis that includes said substantially planar support surface, said neck portion, said one-half spherical structure, said disc, and a portion of said primary spring, and a portion of said secondary spring along said secondary longitudinal axis both to deviate about twenty degrees from vertical from said secondary spring creating an asymmetrical force on said disc from a moment arm distance as between said primary and secondary longitudinal axes resulting in said substantially planar support surface angled in said default first bias position with an initial contact state position, said base outer surface and a portion of said support surrounding sidewall external surface are removably engaged to said human foot side first surface to mount said dual bias omnidirectionally movable substantially planar support surface structure assembly in said orthotic arch support shoe insert insole;
c. an electrode that is solely disposed and affixed upon said substantially planar support surface, wherein said electrode is positioned via said default first bias position initial contact state position to initially contact a portion of a human foot arch skin surface to facilitate a soft contact of the portion of the human foot arch skin surface after which said dual bias omnidirectionally movable substantially planar support surface structure assembly moves into an operational position state via creating a second bias that exerts force on said electrode along said primary longitudinal axis away from said base to operationally exert contact force as between said electrode and the portion of the human foot arch skin surface, even while the portion of the human foot arch skin surface has omnidirectional movement defined as being vertically, rotationally, tilting front to back, and tilting left to right while said second bias keeps the contact force as between said electrode and the portion of the human foot arch skin surface, and during operational removal movement of said human foot side first surface from the portion of the human foot arch skin surface, said dual bias omnidirectionally movable substantially planar support surface structure assembly automatically moves into said default first bias position initial contact state position being ready for a subsequent contact of the portion of the human foot arch skin surface; and
d. control circuitry that is in electrical communication with said electrode, wherein said control circuitry is operative to generate the electrical impulses with particular frequency, phase, pulse versus time amounts, voltage, and ampere levels at user safe low electrical power levels that are less than one-hundred milli-amps and no more than sixty volts.

10. A sensory control system according to claim 9 wherein said electrode and said control circuitry are constructed of a transcutaneous electrical nerve stimulation (TENS) system.

11. A sensory control system according to claim 9 wherein said base inner surface further comprises a primary spring first end portion first recess cavity that partially nests said primary spring first end portion to operationally help keep said primary spring first end portion in position about said primary longitudinal axis and said base inner surface further comprises a secondary spring first end portion second recess cavity that partially nests said secondary spring first end portion to operationally help keep said secondary spring first end portion in position about said secondary longitudinal axis.

12. A sensory control system according to claim 9 wherein said disc first end portion further comprises a primary spring second end portion third recess cavity that partially nests said primary spring second end portion to operationally help keep said primary spring second end portion in position about said primary longitudinal axis and said disc first end portion further comprises a secondary spring second end portion fourth recess cavity that partially nests said secondary spring second end portion to operationally help keep said secondary spring second end portion in position about said secondary longitudinal axis.
